(19) United States
(12) Patent Application Publication
(10) Pub. No.: US 2024/0180556 A1
(43) Pub. Date: Jun. 6, 2024

(54) DEVICES, SYSTEMS, AND METHODS FOR TREATING PULMONARY DISEASE

(71) Applicant: Apreo Health, Inc., Menlo Park, CA (US)

(72) Inventors: James I. Fann, Portola Valley, CA (US); Steven W. Kim, Los Altos, CA (US); Hanson S. Gifford, III, Woodside, CA (US); Douglas S. Sutton, Pacifica, CA (US); Martin L. Mayse, Wayzata, MN (US); Karun D. Naga, Los Altos, CA (US)

(21) Appl. No.: 18/553,788

(22) PCT Filed: Apr. 8, 2022
(86) PCT No.: PCT/US2022/071650

§ 371 Date: Oct. 3, 2023

Related U.S. Application Data
(60) Provisional application No. 63/201,022, filed on Apr. 8, 2021.

(51) Int. Cl. A61B 17/12; A61B 17/00

(52) U.S. Cl. CPC A61B 17/12009; A61B 2017/00022; A61B 2017/00522; A61B 2017/00535; A61B 2017/00809; A61B 2017/00867; A61B 2017/00893; A61B 2017/12018

(57) ABSTRACT

Devices, systems, and methods for treating pulmonary disease are disclosed herein. According to some embodiments, the present technology includes identifying diseased tissue on a lobe of the patient's lung, collapsing the lobe, and percutaneously inserting a device into the patient and a therapeutic element carried by the device. The present technology further includes thoracoscopically delivering the therapeutic element into the patient's chest cavity, administering the therapeutic element on and around at least the diseased tissue, and applying an atraumatic compressive load to and/or restricting expansion of at least a portion of the diseased tissue.

### CROSS-REFERENCE TO RELATED APPLICATION(S)

The present technology claims the benefit of priority to U.S. Provisional Application No. 63/201,022, filed Apr. 8, 2021, which is incorporated by reference herein in its entirety.

### TECHNICAL FIELD

The present technology relates to devices, methods, and systems for treating pulmonary disease.

### BACKGROUND

In the United States, an estimated 15 million adults have chronic obstructive pulmonary disease (COPD), which can be progressive and compromise the ability to breathe. The total number of cases reported may be an underestimate, as there may be as many as 24 million adults living with COPD. Of these, it is estimated that in 2016, 3.5 million Americans have emphysema, which is a subset of COPD (https://www.healthline.com/health/copd/facts-statistics-infographic #Prevalence).

Emphysema is evidenced by the loss of elasticity of the airways and lung tissue, leading to hyperinflation or air trapping in a portion of the lung and decreased overall lung function. Structurally, there is loss of radial integrity of the airways with airway narrowing and destruction of alveolar walls. The result is reduced ability of the lungs to expel air during the exhalation phase of the breathing cycle and to achieve effective oxygen and carbon dioxide exchange. The hyperinflated portion of the lung occupies progressively more space within the chest cavity preventing healthy lung tissue from properly expanding during the inhalation phase of the breathing cycle. This process results in significantly compromised lung function and symptom of shortness of breath.

Lung function in patients with emphysema can be improved by reducing the volume of the diseased lung, typically by surgical resection. This resection permits more effective expansion and function of the healthy portions of the lung. Through an open chest or thoracoscopic approach, lung volume reduction surgery often results in concomitant removal of adjacent healthy lung. While often effective in selected patients, lung volume reduction surgery is traumatic to the patient, and lung injury during the resection process may lead to persistent air leak and other complications. In older frail patients, which is often the case in patients with emphysema, the morbidity and mortality rates can be substantial. Accordingly, lung volume reduction surgery is not commonly performed on patients, even when all other forms of treatment have failed.

One less invasive or endobronchial technique proposed to effect lung volume reduction is one-way valves. A major challenge associated with endobronchial valves is the presence of collateral ventilation from adjacent lung segments in many patients. In these cases, which represent 50-80% of emphysema patients, air can enter collaterally from the adjacent healthy lung tissue leading to persistent hyperinflation of the treated portions of the lung, thereby undermining efficacy of the endobronchial valve. Complex diagnostic technologies are required to determine whether a patient would benefit from an endobronchial valve. Even when a carefully selected patient is treated with a valve there is significant potential for complications like pneumothorax and secondary interventions to deal with migration and fibrosis. Several other methods have been developed such as coils, energy ablation (radiofrequency or microwave), heated water vapor, cryoablation, polymeric injection, and plugs but none have yet demonstrated efficacy. Therefore, there remains a need for safe and effective lung volume reduction.

### SUMMARY

Described herein are devices, systems, and methods for thoracoscopically and/or percutaneously performing a lung volume reduction. Several of the embodiments of the present technology comprise a therapeutic element configured to be applied to an outer surface of a portion of an emphysematous lung to limit and/or prevent expansion of the lung portion to effect lung volume reduction and improve lung function. Methods of the present technology include identifying a diseased portion of the lung for treatment and decompressing or collapsing the lung with single-lung ventilation. A therapeutic element can be inserted percutaneously and then introduced thoracoscopically into the chest cavity at a location that is proximate the identified diseased portion of the lung. The therapeutic element can then be administered to the surface of the diseased portion and secured while the lung remains collapsed. In some embodiments, the therapeutic element is fastened to the lung in a non-traumatic fashion (e.g., without penetrating lung tissue). When the lung is reinflated, the therapeutic element prevents or restricts expansion of the diseased portion, thereby increasing the volume within the chest cavity to permit the healthier portion of the lung to expand.

The subject technology is illustrated, for example, according to various aspects described below, including with reference to FIGS. 1-13B. Various examples of aspects of the subject technology are described as numbered clauses (1, 2, 3, etc.) for convenience. These are provided as examples and do not limit the subject technology.

1. A method of treating a patient for emphysema, the method comprising:
identifying diseased tissue on a patient's lung;
collapsing the lung;
thoracoscopically delivering a therapeutic element into the patient's chest cavity;
while the lung is collapsed, administering the therapeutic element on and around at least the diseased tissue; and
with the therapeutic element, applying an atraumatic compressive load to and/or restricting expansion of at least a portion of the diseased tissue.

2. The method of Clause 1, further comprising reinflating the lung while the therapeutic element applies an atraumatic compressive load to and/or restricts expansion of the at least a portion of the diseased tissue such that a remaining portion of the lung that is not engaged by the therapeutic element inflates to at least a pre-collapsed volume while the diseased tissue is maintained in a substantially collapsed state by the therapeutic element.

3. The method of Clause 1 or Clause 2, wherein collapsing the lung includes collapsing only the one of the right or left lung that includes the diseased tissue identified for treatment.

4. A system for treating a patient having emphysema via thoracoscopic lung volume reduction, the device comprising:
a therapeutic element having a low-profile configuration for thoracoscopic delivery to the patient's chest cavity and an expanded configuration in which the therapeutic element is configured to be disposed on an outer surface of a diseased portion of a patient's lung; and
an applicator configured to manipulate the therapeutic element within the chest cavity to place the therapeutic element in contact with the diseased portion of the patient's lung when the lung is collapsed,
wherein the therapeutic element is configured to apply a compressive load to and/or restrict expansion of the diseased portion of the patient's lung, and
wherein the therapeutic element is configured to, upon reinflation of the patient's lung, restrict airflow to the diseased portion of the patient's lung, thereby achieving a lung volume reduction, and
wherein interaction between the patient's lung and the therapeutic element produces a contact force and the therapeutic element is configured to distribute the contact force so as to maintain perfusion to the diseased portion of the patient's lung and/or minimize tension in the at least a diseased portion of the patient's lung.

5. The system of Clause 4, wherein the therapeutic element comprises a flexible film.

6. The system of any one of the previous Clauses, wherein the applicator comprises a plurality of arms and is configured to transform the therapeutic element from the low-profile configuration to the expanded configuration while the therapeutic element is disposed within the chest cavity.

7. The system of any one of the previous Clauses, wherein the therapeutic element comprises a flexible film, and wherein, when the film is in the low-profile configuration, the film is rolled up.

8. The system of Clause 7, wherein the applicator is configured to transform the film from the low-profile configuration to the expanded configuration by unrolling the film.

9. The system of Clause 7, wherein the applicator comprises a first arm and a second arm, and wherein the first arm is configured to unroll the film from and the second arm is configured to stabilize the film.

10. The system of any one of the previous Clauses, wherein the therapeutic element comprises a tubular sleeve.

11. The system of any one of the previous Clauses, wherein the therapeutic element is configured to be wrapped around the lung at least one once.

12. The system of any one of the previous Clauses, wherein the therapeutic element is configured to adhere to itself.

13. The system of any one of the previous Clauses, wherein the therapeutic element is configured to be wrapped around the lung at least once and secured via a fastener.

14. The system of Clause 13, wherein the therapeutic element is a film and the fastener comprises a thicker portion of the film that generates friction with the lung tissue having a magnitude that is greater than a magnitude of friction generated between the rest of the film and the lung tissue.

15. The system of Clause 14, wherein the therapeutic element is configured to be positioned on a lobe of the lung such that the fastener is aligned with a lower edge of the lobe.

16. The system of any one of the previous Clauses, further comprising a fastener, and wherein the fastener is configured to secure the therapeutic element to itself such that the lung tissue is compressively engaged and/or captured within the therapeutic element without the fastener directly engaging the lung tissue, and wherein the fastener comprises at least one of: an adhesive, a mechanical fastener, and/or a weld in situ.

17. The system of any one of the previous Clauses, wherein, when the therapeutic element is positioned over the diseased portion of the lung, the therapeutic element has an end portion that is open and exposing the diseased portion to the pleural cavity, and wherein the system further includes a band configured to be positioned around the open end portion to close the end portion, thereby limiting or preventing the passage of air between the pleural cavity and the diseased portion and limiting or preventing herniation of the diseased portion.

18. The system of any one of the previous Clauses, wherein the therapeutic element is configured to be substantially impermeable to air to seal the lung tissue and prevent leaks.

19. The system of any one of the previous Clauses, wherein the therapeutic element comprises a film and a substance comprising a liquid or gel.

20. The system of Clause 19, wherein the substance comprises a biological or polymeric resin and/or glue.

21. The system of Clause 19 or Clause 20, wherein the substance is configured to be applied to fill interstitial voids between the film and healthy and/or diseased lung tissue to improve attachment and/or sealing between the therapeutic element and the lung tissue.

22. The system of any one of the previous Clauses, wherein the liquid or gel substance is configured to be applied outside the film (i.e., on the other side of the film from the tissue-facing side), and wherein the liquid or gel substance is configured to cure and set the film in a substantially rigid form to restrict expansion of the at least a diseased portion of the patient's lung.

23. The system of any one of the previous Clauses, wherein the therapeutic element is configured to secure the at least a diseased portion of the patient's lung in a substantially flat configuration (i.e., length and/or width are substantially greater than the thickness), and wherein the substantially flat configuration minimizes the contact force and attenuates tension in the lung tissue.

24. The system of any one of the previous Clauses, wherein the therapeutic element comprises a polymer coating configured to adhere to the diseased portion of the patient's lung, and wherein the polymer coating is configured to be sprayed onto the patient's lung when the patient's lung is in a collapsed state.

25. The system of Clause 24, wherein the therapeutic element further comprises a biocompatible film, and wherein the film is configured to be applied to and/or wrapped around the polymer coating.

26. The system of Clause 25, wherein the film is configured to be a buttress for the polymer coating to enhance fixation and/or sealing of the therapeutic element.

27. The system of any one of the previous Clauses, wherein the therapeutic element includes a therapeutic agent and is configured to deliver the therapeutic agent to the lung tissue while the therapeutic element is secured to the lung tissue.

28. The system of Clause 25, wherein the therapeutic agent is at least one of any anti-inflammatory, an antibiotic, and/or an anesthetic.

29. The system of Clause 27 or Clause 28, wherein the therapeutic element comprises a polymeric coating or film that is loaded with the therapeutic agent.

30. The system of any one of the previous Clauses, wherein the therapeutic element comprises a biocompatible material, wherein the biocompatible material: (a) is either non-degradable, partially-degradable or fully-degradable material, (b) comprises a polymeric or biological material, and/or (c) comprises at least one of silicone, polyurethane, PTE, PTFE, polyester, nylon, PLGA, PLA, PCL (polycaprolactone), PLCL and combinations thereof.

31. The system of any one of the previous Clauses, wherein the applicator and/or therapeutic element is configured for thoracoscopic delivery through at least one port in the chest wall.

32. The system of any one of the previous Clauses, wherein the system includes a thoracoscope camera.

33. The system of any one of the previous Clauses, wherein the applicator and/or therapeutic element is configured for thoracoscopic delivery via a single port.

34. The system of any one of the previous Clauses, wherein the applicator and/or therapeutic element is configured for thoracoscopic delivery via a multiple ports.

35. The system of any one of the previous Clauses, wherein the therapeutic element comprises a mesh.

36. The system of Clause 35, wherein the therapeutic element further comprises a polymer film and/or adhesive configured to secure the mesh to the diseased portion of the lung.

37. The system of any one of the previous Clauses, wherein the therapeutic element comprises a structure configured to be disposed on and around the diseased portion of the patient's lung, wherein the structure includes a channel configured to receive a fluid therethrough.

38. The system of Clause 37, wherein a size and/or shape of the structure can be adjusted by controlled delivery of the fluid to and withdrawal of the fluid from the structure, thereby enabling in-situ customization of the therapeutic element.

39. The system of Clause 37 or 38, wherein upon initial placement of the therapeutic element against the lung tissue, the channel is configured to receive a volume the fluid to confirm apposition of the therapeutic element and the lung tissue and/or a contact force between the therapeutic element and the lung tissue, and, after the fluid is evacuated from the channel, the channel is configured to receive a volume of a substance equal to the volume of fluid, wherein the substance is configured to solidify in-situ and take a permanent shape consistent with the confirmed apposition and/or contact force.

40. The system of any one of Clauses 37 to 39, wherein the structure is configured to assume a triangular two-dimensional (2D) shape and/or a conical and/or pyramidal three-dimensional (3D) shape when the fluid is delivered to the channel to approximate the native geometry of the portion of the lung contained by the therapeutic element, and wherein the 2D and/or 3D shape enables the therapeutic element to prevent or limit twisting of the contained portion of the lung upon re-inflation of the remainder of the lung, and wherein the therapeutic element is configured to limit or prevent constriction of the vasculature perfusing the contained portion of the lung.

41. The system of any one of Clauses 37 to 40, further comprising a substance configured to be delivered into the channel and solidify within the channel to rigidize the structure in a desired shape.

42. The system of any one of the previous Clauses, further comprising a sensor, incorporated into or independent of the therapeutic element, wherein the sensor is configured to monitor perfusion to the portion of the lung contained by the therapeutic element to reduce a risk of ischemia and necrosis of the contained portion of the lung.

43. The system of any one of the previous Clauses, further comprising an imaging device for monitoring the patient following placement of the therapeutic element.

44. The system of Clause 43, wherein the imaging device is configured to obtain image data via computed tomography (CT), magnetic resonance imaging (MRI), positron emission tomography (PET), ultrasound, X-ray, or combinations thereof.

45. The system of Clause 44, wherein the therapeutic element includes an enhancing agent for enhancing visualization of the therapeutic element when imaged by the imaging device.

46. The system of any one of the previous Clauses, wherein the therapeutic element is configured to have a triangular two-dimensional (2D) shape and/or a conical and/or pyramidal three-dimensional (3D) shape when in an expanded configuration and restricting expansion of the lung.

47. A method of treating a patient for emphysema, the method comprising:
identifying diseased tissue on a lobe of the patient's lung;
collapsing the lobe;
percutaneously inserting a device into the patient, the device carrying a therapeutic element;
thoracoscopically delivering the therapeutic element into the patient's chest cavity;
positioning the therapeutic element on and around at least the diseased tissue; and
applying an atraumatic compressive load to and/or restricting expansion at least a portion of the diseased tissue.

48. An apparatus for treating a patient having emphysema via thoracoscopic lung volume reduction, the apparatus comprising:
a therapeutic element in a reduced configuration for thoracoscopic delivery to the patient's chest cavity; and
an applicator configured to expand the therapeutic element and subsequently place the therapeutic element in contact with at least a diseased portion of the patient's lung when the lung is collapsed,
wherein the therapeutic element is configured to apply a compressive load to or restrict expansion of the at least a diseased portion of the patient's lung, and
wherein the therapeutic element is configured to upon reinflation of the patient's lung restrict airflow to the at least a diseased portion of the patient's lung, thereby achieving a lung volume reduction, and
wherein interaction between the patient's lung and the therapeutic element produces a contact force and the therapeutic element comprises a structure for distributing the contact force so as to maintain perfusion to the at least a diseased portion of the patient's lung and/or minimize tension in the at least a diseased portion of the patient's lung.

49. The apparatus of Clause 48, wherein the therapeutic element comprises a flexible film.

50. The apparatus of any one of the previous Clauses, wherein the film in the reduced configuration comprises the film wrapped around a spool.

51. The apparatus of any one of the previous Clauses, wherein the applicator is configured to release the film from the reduced configuration using independent or coordinated arms; wherein the independent or coordinated arms comprise a first arm and a second arm and wherein the first arm is configured to roll out the film and the second arm is configured to stabilize the film.

52. The apparatus of any one of the previous Clauses, wherein the flexible film comprises a cylindrically shaped polymer sleeve.

53. The apparatus of any one of the previous Clauses, wherein the film is configured to be wrapped around the lung two or more times and secured by adhering to itself.

54. The apparatus of any one of the previous Clauses, wherein the film is configured to be administered by the clinical/surgical operator in a form or shape that is matched to the particular needs of the patient (i.e., “in situ customization”).

55. The apparatus of any one of the previous Clauses, wherein the film is configured to be substantially impermeable to air to seal the lung tissue and prevent leaks.

56. The apparatus of any one of the previous Clauses, wherein the film is configured to be wrapped around the lung at least once and secured through a fastener.

57. The apparatus of any one of the previous Clauses, wherein the fastener comprises a thicker portion of the film to create increased friction.

58. The apparatus of any one of the previous Clauses, wherein the fastener comprises the film being secured at an anatomic level of the lung such as the lobar fissure using a thicker portion of the film at one edge (the lower edge as in the case of the lobar fissure) as it is wrapped and adheres to itself.

59. The apparatus of any one of the previous Clauses, wherein the fastener comprises various fastening means for securing the film to itself (instead of securing to the tissue) such that the lung tissue is compressively engaged/captured within the wrapped film. wherein fastening means comprises at least one of adhesive, mechanical fasteners (e.g., clamp, staple, tape, elastic band/compressive sleeve), welding in situ (e.g., laser).

60. The apparatus of any one of the previous Clauses, wherein the therapeutic element comprises a film and a liquid or gel substance.

61. The apparatus of any one of the previous Clauses, wherein the liquid or gel substance comprises a biological or polymeric resin or glue.

62. The apparatus of any one of the previous Clauses, wherein the liquid or gel substance is configured to be applied to fill interstitial voids between the film and lung tissue so as to optimize attachment and/or sealing between the therapeutic element and the lung tissue.

63. The apparatus of any one of the previous Clauses, wherein the liquid or gel substance is configured to be applied outside the film (i.e., on the other side of the film from the tissue-facing side); wherein the liquid or gel substance is configured to cure and set the film in a substantially rigid form to restrict expansion of the at least a diseased portion of the patient's lung.

64. The apparatus of any one of the previous Clauses, wherein the therapeutic element is configured to secure the at least a diseased portion of the patient's lung in a substantially flat configuration (i.e., length and/or width are substantially greater than the thickness); wherein in the substantially flat configuration minimizes the contact force and attenuates tension in the lung tissue.

65. The apparatus of any one of the previous Clauses, wherein the therapeutic element comprises a polymer coating configured to adhere to the at least a diseased portion of the patient's lung; wherein the polymer coating is configured to be sprayed on to the patient's lung when in a collapsed state.

66. The apparatus of any one of the previous Clauses, wherein the therapeutic element further comprises a biocompatible film; wherein the film is configured to be applied to and/or wrapped around the polymer coating; wherein the film is configured to be a buttress for the polymer coating to enhance fixation and/or sealing of the therapeutic element.

67. The apparatus of any one of the previous Clauses, wherein the therapeutic element is substantially impermeable to air/gas to prevent air leaks from the lung tissue.

68. The apparatus of any one of the previous Clauses, wherein the therapeutic element is configured to deliver a therapeutic agent to the lung tissue.

69. The apparatus of any one of the previous Clauses, wherein the therapeutic agent is at least one of any anti-inflammatory, an antibiotic, or an anesthetic.

70. The apparatus of any one of the previous Clauses, wherein the therapeutic element comprises a polymeric coating or film loaded with the therapeutic agent.

71. The apparatus of any one of the previous Clauses, wherein the therapeutic element comprises a film having a portion (the open end) that is configured to be secured by a constricting band to create a closed end for airtight seal and to ensure that there is no herniation of the collapsed lung.

72. The apparatus of any one of the previous Clauses, wherein the therapeutic element is comprised of a biocompatible material; wherein the biocompatible material may be non-degradable, partially-degradable or fully-degradable material; wherein the biocompatible material comprises a polymeric or biological material; wherein the biocompatible material comprises at least one of silicone, polyurethane, PTE, PTFE, polyester, nylon, PLGA, PLA, PCL (polycaprolactone), PLCL and combinations thereof.

73. The apparatus of any one of the previous Clauses, wherein the apparatus is configured for thoracoscopic delivery via at least one port.

74. The apparatus of any one of the previous Clauses, wherein the apparatus comprises a thoracoscope camera.

75. The apparatus of any one of the previous Clauses, wherein the apparatus is configured for thoracoscopic delivery via a single port (i.e., uniport).

76. The apparatus of any one of the previous Clauses, wherein the apparatus is configured for thoracoscopic delivery via a multiple ports (i.e., multiport).

77. The apparatus of any one of the previous Clauses, wherein the apparatus is configured for thoracoscopic delivery via a single port (i.e., uniport).

78. The apparatus of any one of the previous Clauses, wherein the therapeutic element comprises a gauze or mesh-like material with perforations configured for application to or around the diseased portion.

79. The apparatus of any one of the previous Clauses, wherein the therapeutic element further comprises a polymer film or adhesive configured to secure the gauze or mesh-like material to the lung.

80. The apparatus of any one of the previous Clauses, wherein the therapeutic element comprises a wrap or envelope outer structure for application to the diseased portion of the patient's lung, wherein the wrap or envelope comprises a hydro-skeleton structure.

81. The apparatus of any one of the previous Clauses, wherein the hydro-skeleton structure is configured for in-situ customization and proper sizing and fit of the wrap or envelope;

82. The apparatus of any one of the previous Clauses, wherein upon initial placement of the therapeutic element against the lung tissue, the hydro-skeleton is configured to first receive a volume of water or some other liquid to confirm apposition and contact force of the therapeutic element and, after the water is evacuated from the hydro-skeleton, subsequently a volume of liquid or gel polymer equal to the volume of water, wherein the liquid or gel polymer is configured to cure in-situ and take a permanent shape consistent with the confirmed apposition and contact force.

83. The apparatus of any one of the previous Clauses, wherein the hydro-skeleton comprises a triangular (in two dimensions) or conical/pyramidal (in three dimensions) shape when instilled with a liquid or polymer to maximize contact with and minimize twisting of the compressed portion of the lung upon re-inflation of the remainder of the lung; wherein the hydro-skeleton is configured to minimize the constriction of the artery and vein perfusing the lung.

84. The apparatus of any one of the previous Clauses, wherein the hydro-skeleton is configured to be instilled with a water or saline solution to test the compression properties of the wrap or envelope outer structure against the lung tissue; after assuring the proper configuration, the water or saline solution may be replaced by a material, such as a polymer, that can solidify providing conformity and rigidity to the hydro-skeleton of the wrap or envelope.

85. The apparatus of any one of the previous Clauses, wherein the apparatus further comprises a flow sensor, such as a doppler probe, incorporated into or independent of the therapeutic element to monitor and ensure adequacy of perfusion to the compressed lung during the application of the therapeutic element to minimize risk of ischemia and necrosis of the compressed lung.

86. A system for treating a patient having emphysema via thoracoscopic lung volume reduction, the system comprising:
an apparatus as described herein, the apparatus comprising a therapeutic element and an applicator; and
an imaging modality for monitoring the patient following surgery.

87. The system of Clause 86, wherein the imaging modality comprises computed tomography (CT), magnetic resonance imaging (MRI), positron emission tomography (PET), ultrasound, X-ray or combinations thereof.

88. The system of Clause 86, wherein the system further comprises a sensor, marker and/or dye for enhancing visualization via the imaging modality; where the therapeutic element comprises the sensor, marker and/or dye.

89. A method of treating a patient for emphysema, the method comprising:
identifying for treatment a portion of diseased tissue on the patient's lung;
collapsing the lobe of the patient that corresponds to the portion of diseased tissue;
percutaneously inserting into the patient an apparatus carrying a therapeutic element;
thoracoscopically delivering the therapeutic element into the patient's chest cavity;
administering the therapeutic element to the collapsed lobe; and
applying an atraumatic compressive load to and/or restricting expansion of at least a portion of the collapsed lobe.

90. A device for treating a patient having emphysema via thoracoscopic lung volume reduction, the device comprising:
a therapeutic element having a low-profile configuration for thoracoscopic delivery to the patient's chest cavity and an expanded configuration in which the therapeutic element is configured to be disposed on an outer surface of a diseased portion of a patient's lung; and
wherein the therapeutic element is configured to apply a compressive load to and/or restrict expansion of the diseased portion of the patient's lung, and
wherein the therapeutic element is configured to, upon reinflation of the patient's lung, restrict airflow to the diseased portion of the patient's lung, thereby achieving a lung volume reduction, and
wherein interaction between the patient's lung and the therapeutic element produces a contact force and the therapeutic element is configured to distribute the contact force so as to maintain perfusion to the diseased portion of the patient's lung and/or minimize tension in the at least a diseased portion of the patient's lung.

91. The device of Clause 90, wherein the therapeutic element comprises a cover.

92. The device of Clause 90 or Clause 91, wherein the therapeutic element comprises a fastener.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates the lungs of a human patient suffering from emphysema.

FIG. 2 shows a therapeutic element of the present technology positioned on and containing a diseased portion of the lungs.

FIGS. 3A-3C show a method for installing a therapeutic element of the present technology on a collapsed lung.

FIG. 4A shows a film configured in accordance with several embodiments of the present technology.

FIG. 4B shows the film of the FIG. 4A loaded on an applicator in a partially rolled, partially deployed configuration, and configured in accordance with several embodiments of the present technology.

FIG. 4C shows the film of the FIG. 4A loaded on an applicator in an unrolled configuration in accordance with several embodiments of the present technology.

FIG. 5A shows a thoracoscopic tool holding stabilizing a portion of a collapsed lung in accordance with several embodiments of the present technology.

FIG. 5B shows the applicator of FIGS. 4B and 4C wrapping a therapeutic element around a diseased portion of the lung in accordance with several embodiments of the present technology.

FIG. 6 shows a therapeutic element and a fastener positioned over a collapsed, diseased portion of the lung and configured in accordance with several embodiments of the present technology.

FIG. 7A shows a therapeutic element and a fastener positioned over a collapsed, diseased portion of the lung and configured in accordance with several embodiments of the present technology.

FIG. 7B shows a therapeutic element and a fastener positioned over a collapsed, diseased portion of the lung and configured in accordance with several embodiments of the present technology.

FIGS. 8A and 8B depict a therapeutic element of the present technology positioned on a collapsed lung in a first and second configuration, respectively.

FIGS. 9A and 9B are front and side views of a therapeutic element configured in accordance with several embodiments of the present technology.

FIG. 10 shows the therapeutic element shown in FIGS. 9A and 9B containing a portion of a diseased lung in accordance with several embodiments of the present technology.

FIG. 10A illustrates a therapeutic element configured in accordance with several embodiments of the present technology.

FIG. 10B illustrates a therapeutic element configured in accordance with several embodiments of the present technology.

FIG. 11 illustrates a therapeutic element configured in accordance with several embodiments of the present technology.

FIG. 12 illustrates a therapeutic element and an expansion member positioned in a chest cavity and configured in accordance with several embodiments of the present technology.

FIGS. 13A and 13B show a method for administering a therapeutic element to a diseased portion a lung in accordance with several embodiments of the present technology.

### DETAILED DESCRIPTION

In patients with emphysema, not only are the diseased portions of the lung inactive, thereby reducing the functional capacity of the lung, but the diseased tissue also occupies valuable space in the chest cavity, thereby undermining the function of the remaining healthy lung tissue. It has been demonstrated that collapse or resection of a portion of the lung can result in lung volume reduction and have a therapeutic benefit on the patient's lung function, thereby improving the patient's symptoms and quality of life. This lung volume reduction has been demonstrated surgically and via endobronchial procedures to significantly improve patients' pulmonary function as measured by forced expiratory volume (FEV), the six-minute walk test, and St. George's Respiratory Questionnaire (SGRQ). These surgical and interventional approaches, however, have suffered from limited anatomic/physiologic applicability and/or high complication rates. Some proposed methods include placing a compressive device over a diseased portion of the lung to selectively restrict expansion of the diseased lung, thereby increasing the volume within the chest cavity to permit the healthier portion of the lung to expand. These proposals have been unsuccessful as they fail to address how to engage, constrict, and adhere the compressive device to the diseased lung without creating a risk of lung injury, perforation, persistent leakage, and/or necrosis that would negate any therapeutic benefit. For example, some of the proposed devices indiscriminately apply a high compressive force to the diseased tissue to achieve better anchoring and sealing without accounting for the fact that such high forces often restrict blood flow to the associated lobe and, accordingly, cause necrotic injury to the lungs. Furthermore, the contact forces on the diseased lobe and surrounding tissue can cause tension in the fragile lung tissue, which creates risk of perforation and pneumothorax.

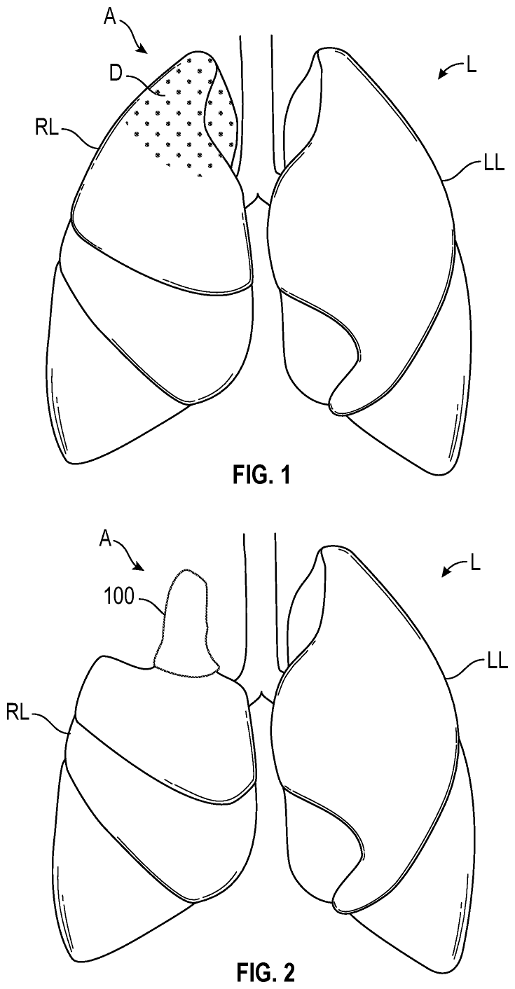

The devices, systems, and methods of the present technology address the foregoing challenges and provide effective lung volume reduction in a manner that is less traumatic than surgical resection, decreases the risk of persistent air leakage or pneumothorax, and overcomes the effects of collateral ventilation. In particular, the present technology includes one or more therapeutic elements that are configured to be disposed on the emphysematous lung tissue to restrict expansion of the diseased tissue while preventing rupture or perforation. FIG. 1, for example, depicts the lungs L of a human patient having a hyperinflated diseased portion D at the apical portion A of the right lung RL. FIG. 2 shows a therapeutic element 100 of the present technology positioned over the diseased portion D and selectively restricting or preventing the diseased portion D from inflating and/or expanding. By constraining the diseased portion, the therapeutic element 100 increases the open volume within the chest cavity to permit the healthier portion of the lung to expand. While the therapeutic elements and associated devices of the present technology are shown and described being used to restrict, capture, constrain, and/or compress the apical portion A of the superior lobe of the right lung RL, the present technology can be utilized to restrict, capture, constrain, and/or compress any portion and/or any lobe of the right lung RL and the left lung LL.

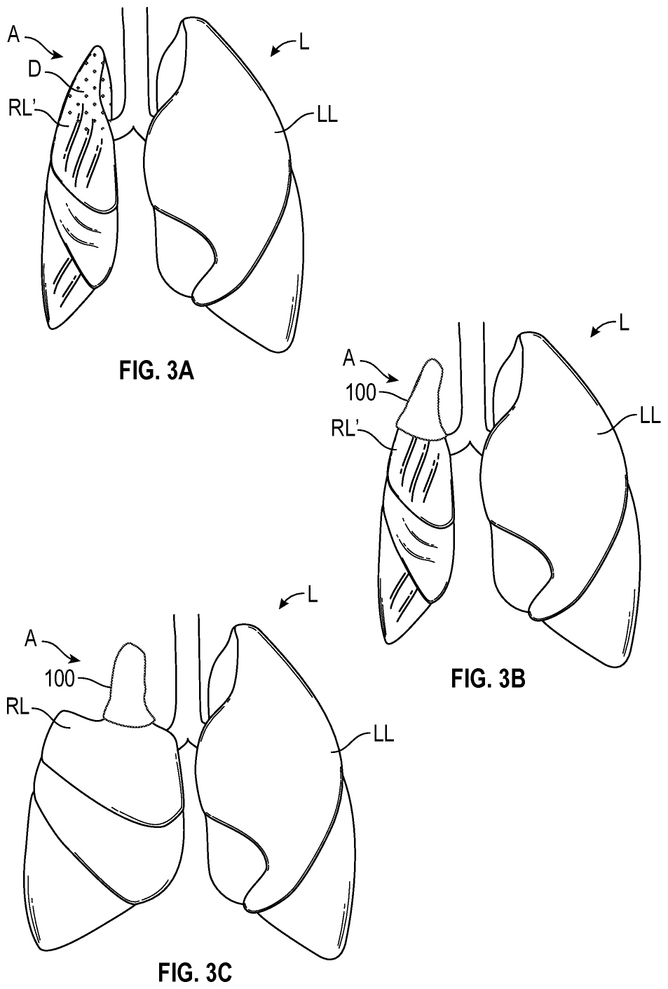

The therapeutic element 100 can have a low-profile configuration for thoracoscopic delivery to the patient's chest cavity and an expanded configuration in which the therapeutic element 100 is configured to be disposed on and around an outer surface of a diseased portion of the lung. As shown in FIGS. 3A-3C, in some embodiments the therapeutic element 100 can be applied to the diseased tissue while the affected lung is collapsed. As shown in FIG. 3A, with the patient under general anesthesia, single lung ventilation can be used to selectively collapse the lung identified for treatment (in this case, the right lung RL). The therapeutic element 100 can then be inserted percutaneously and introduced thoracoscopically into the chest cavity at a location proximate the diseased portion of the lung. The therapeutic element 100 can be configured for thoracoscopic delivery via a single port or multiple ports. As shown in FIG. 3B, the therapeutic element 100 is then administered to an outer surface of the collapsed, diseased portion of the lung and secured in place. As depicted in FIG. 3C, the therapeutic element 100 constrains the diseased portion of the lung such that upon reinflation, more air flows into the healthy portion of the lung than into the constricted diseased portion. This allows the healthy portion of the lung to fully function without interference from the diseased lung, which is maintained in a partially or fully collapsed state by the therapeutic element 100.

As previously mentioned, one objective of the present therapy is to avoid or minimize injury to the lung. Stress or tension from a compressive load has the potential for adverse consequences to the patient. For example, a high compressive or constrictive force exerted by the therapeutic element 100 may compromise arterial and/or venous flow and result in tissue necrosis. Such necrosis may incite an overwhelming inflammatory response leading to significant patient morbidity (e.g., infection) and potential mortality. Perforation or rupture of the lung tissue as a result of tissue contact stresses associated with the compressive load can also cause complications. This risk can be substantial due to the frail state of lung parenchyma and the high differential airspace tension at the interface of the compressed lung and the un-compressed lung which is exacerbated when the tissue is diseased. As discussed herein, the therapeutic element 100 is configured to apply a restrictive, compressive, and/or constraining force to the diseased tissue at a magnitude and distribution that restricts airflow and anchors the therapeutic element 100 while still allowing perfusion to the lung tissue and limiting any disturbance to existing tension differentials across the tissue.

Another objective of the present technology is to provide a durable implant with a durable treatment effect. Durability can be especially difficult to achieve in the dynamic environment of the lungs. For example, the average person has approximately 16 respirations per minute, 960 respirations per hour, 23,000 respirations per day, and 8.4 million respirations per year. The therapeutic element 100 is configured to withstand many respiration cycles without mechanical fatigue or migration. For example, the therapeutic element 100 is configured to be implanted for at least one month, two months, three months, four months, five months, six months, seven months, eight months, nine months, 10 months, 11 months, one year, 18 months, two years, three years, four years, or five years.

Still another objective is to seal the lung tissue and prevent air leakage into the captured portion of the lung. As a result, the therapeutic element 100 is configured to be substantially impermeable to air. The therapeutic element 100 may also comprise a fastener to both seal and secure the therapeutic element 100 in positioned over the captured portion of the lung, as detailed herein.

Still another objective is to accommodate variations from patient to patient. These variations may be due to differences in patient anatomy (i.e., lobe size) or differences in the presentation or manifestation of disease in the anatomy. The therapeutic element 100 can be configured for use across a variety of patient anatomies. As discussed below, in some embodiments the therapeutic element 100 has a size and/or shape that can be customized in situ.

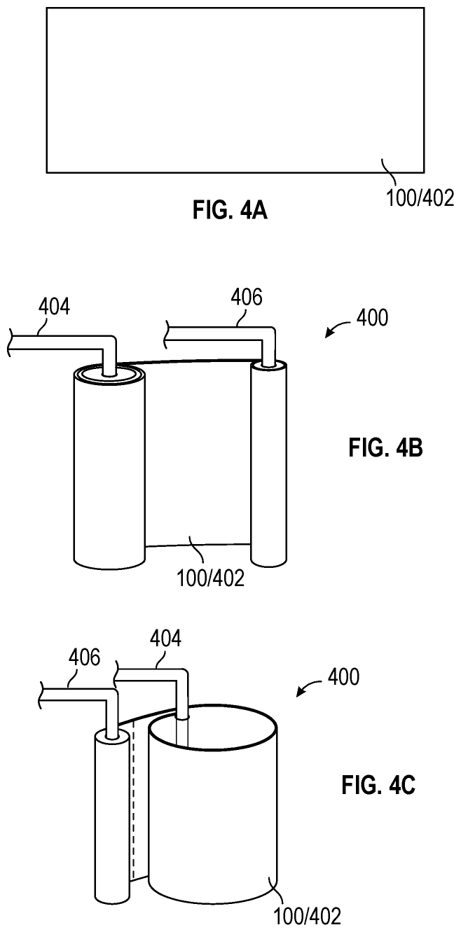

In some embodiments, the therapeutic element 100 comprises a biocompatible film that is configured to be wrapped around the diseased portion of the lung. FIG. 4A, for example, shows a film 402 in a laid-flat configuration. The film 402 can comprise a thin, flexible sheet having a length sufficient to be wrapped around the lung once, twice, or several times, and any amount therebetween (e.g., 1.1 times, 1.2 times, 2.5 times, etc.). In some embodiments, the film 402 can be secured by adhering to itself (similar to the mechanism of cellophane wrap). In these and other embodiments, the film 402 can be secured with a mechanical fastener and/or a solidifying and/or binding agent (as detailed herein). For example, according to some embodiments, an adhesive is applied topically or incorporated into the film 402 (like an adhesive tape) to secure the film 402 to the surface of the lung. In some embodiments, the film 402 can be secured to prevent slippage at a level of the lung (such as the lobar fissure) using a thicker portion of the film 402 at its edge as it is wrapped around the portion of the lung and adheres to itself. When the lung is reinflated, the film 402 exerts a restrictive and/or compressive force on the captured portion such that the captured portion remains collapsed as the remaining portions of the lung expand.

In some embodiments, the film 402 (or any therapeutic element 100 disclosed herein) is configured to be positioned over, around, and/or on the diseased portion of the lung using an applicator configured for minimally invasive deployment of the film 402 within the chest cavity. The film 402 can be pre-loaded in the applicator, or may come separate from the applicator and require loading by an operator prior to use.

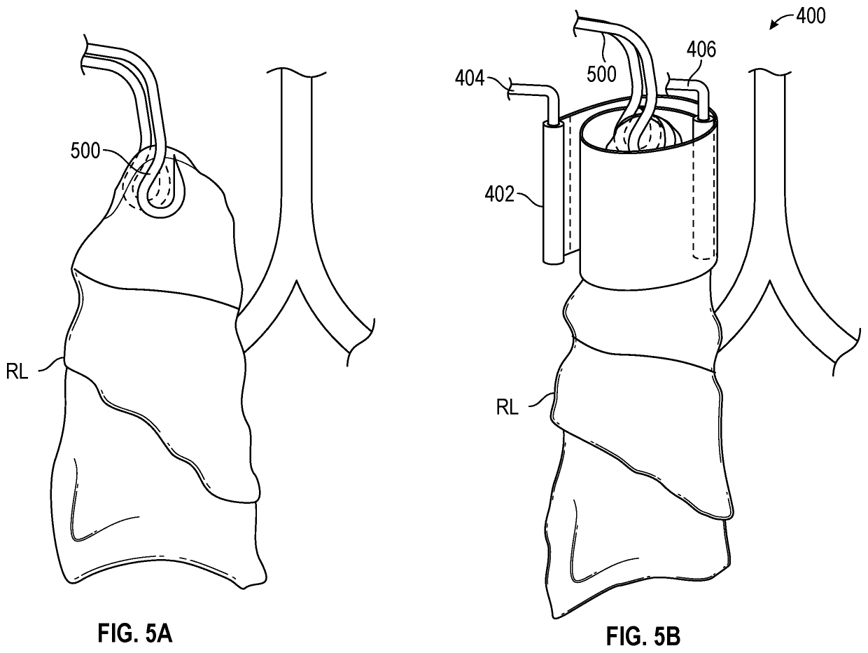

FIGS. 4A and 4B show an example applicator 400 configured in accordance with several embodiments of the present technology. The applicator 400 can comprise a first arm 404 configured to be coupled to a first end portion of the film 402 and a second arm 406 configured to be coupled to a second end portion of the film 402. One or both of the first and second end portions can be rolled around a distal end portion of a corresponding one of the first and second arms 404, 406. In some embodiments, one or both of the first and second arms 404, 406 can have a bend along its respective distal end portion, and the rolled portion(s) of the film 402 can be disposed distal of the bend. The first and second arms 404, 406 can be independently manipulated to unroll the film 402 and wrap the film 402 around the diseased portion (see FIG. 5B). In some embodiments, one of the first or second arms 404, 406 stabilizes the film 402 and the other of the first or second arms 404, 406 unrolls the film 402. In FIGS. 4A and 4B, for example, the first arm 404 stabilizes the film 402 while the second arm 406 moves around the first arm 404 to unroll the portion of the film 402 on the first arm 404.

In some embodiments, one or more auxiliary tools can be utilized to stabilize the selectively deflated lung L before or while the film 402 is wrapped or otherwise engaged by the therapeutic element 100 and/or film 402. For example, as shown in FIG. 5A, a conventional endoscopic grasper 500 may be used to stabilize the selectively deflated lung L. In FIG. 5A, the endoscopic grasper 500 is shown stabilizing the apical portion of the right lung RL. Additionally or alternatively, other stabilizing elements may be utilized to stabilize the deflated lung, such as a vacuum to exert negative pressure on the tissue.

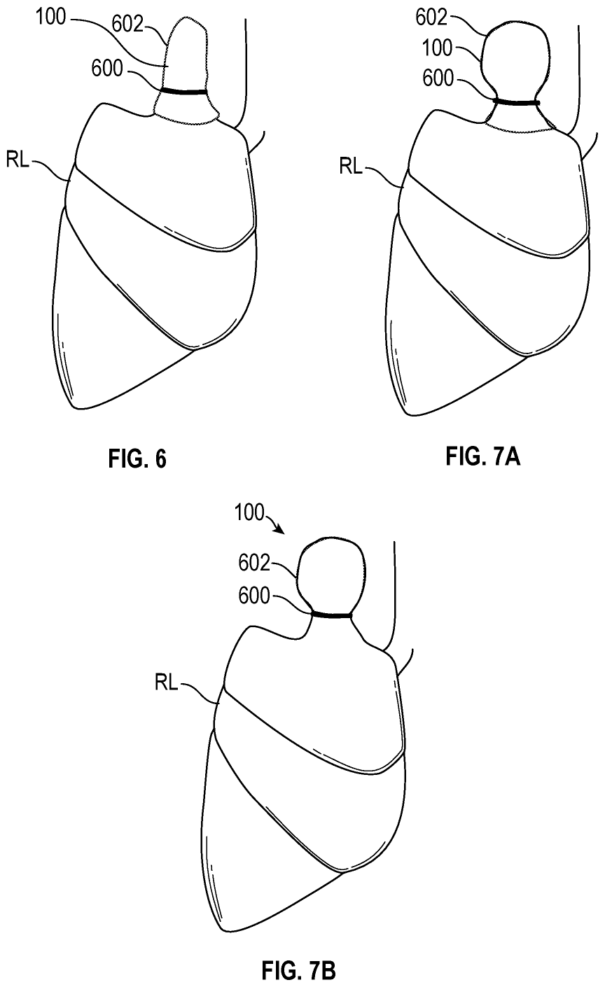

In any of the embodiments disclosed herein, the therapeutic element 100 may be secured to itself and/or to the lung via one or more fasteners. As used herein, a “fastener” can be a standalone device that is coupled to the therapeutic element in situ, a component and/or substance that is separate from and applied and/or coupled to the tissue covering portion (e.g., a film, a cover, etc.), or it may be a portion of the covering portion of the therapeutic element (e.g., a self-adhering surface, a thicker portion of the cover, etc.). The cover and/or covering portions of the present technology may be substantially impermeable to fluids. FIG. 6 shows a therapeutic element 100 comprising a cover 602 secured in place over the captured lung tissue via a fastener 600. In some embodiments, the fastener 600 is a band. In some embodiments, the band comprises a resilient and/or elastic material that exerts a chronic inward force on the therapeutic element 100 and the underlying lung tissue. According to some embodiments, the band can be slid onto the diseased lobe from the apex A. In some embodiments, the band can be opened to be positioned around the therapeutic element 100 and lung tissue (e.g., the band can be assembled onto the diseased lobe), and can be configured to ratchet down to adjust the circumference of the band. In some embodiments, the band comprises a remotely controlled constrictor. Telemetric control of the band can advantageously enable a more gradual compression and/or constriction of the lung tissue, which may be better tolerated by the tissue. In any case, an auxiliary stabilizing tool (as discussed herein) can be utilized to stabilize the lung tissue and/or therapeutic element 100 while the band is put in place. In any of the embodiments disclosed herein, the band can be adjusted via a single port.

In some embodiments, the fastener is configured to secure the therapeutic element to itself such that the lung tissue is compressively engaged and/or captured within the therapeutic element without the fastener directly engaging the lung tissue. In such embodiments, the fastener can comprise at least one of: an adhesive, a mechanical fastener, and/or a weld in situ. According to some aspects of the technology, the system can be configured to secure the tissue in a collapsed state by twisting or otherwise contorting the diseased tissue while in a collapsed state. In some embodiments, system may be configured to deliver thermal energy to weld the collapsed lung to itself. In some embodiments, the thermal energy may be steam. According to certain embodiments, the system can include an applicator with opposing treatment surfaces that can be brought together on either side of the tissue to sandwich the tissue therebetween. The surfaces can be configured to deliver energy (e.g., RF, microwave, HIFU, laser, etc.) to the tissue to weld the tissue to itself. In some of such embodiments, the treatment surfaces can be cooled (e.g., via circulated saline) to control the depth and effect of the energy delivery. In other embodiments, the system can comprise a plurality of bioresorbable tethers configured to be implanted across the lung tissue to gently hold the tissue in a more collapsed state.

As shown in FIGS. 7A and 7B, in some situations it may be beneficial to leverage the diseased properties of the tissue to secure the therapeutic element 100 to the lung. As previously described, the diseased portion intended for capture by the therapeutic element 100 traps air. According to some methods, for example as shown in FIG. 7A, a therapeutic element 100 (or cover 602 thereof) can be positioned on a diseased portion of the lung while the lung is in a collapsed state and a fastener 600 can be slid down over the collapsed apex to a position that is at or near the proximal edge of the cover 602 while still being positioned on the cover 602. A small amount of air can then be let back into a portion of the diseased lobe that is distal to the fastener 600 to expand that portion to a minimum cross-sectional dimension that is greater than the cross-sectional dimension of the fastener 600. Because of the diseased nature of the tissue, the air received by the diseased portion remains in the diseased portion. As a result, the slightly expanded, more bulbous portion of the diseased tissue prevents the fastener 600 from slipping distally off the lobe. Because the fastener 600 is not fixed to the cover 602, a small amount of movement is allowed between the fastener 600 and the cover 602, and between one or both of the fastener 600 and the cover 602 and the lung tissue. This ability of the fastener 600 and the cover 602 to move relative to each other and/or relative to the tissue can advantageously reduce the contact forces on the lung tissue and prevent tearing the lung tissue and/or a pneumothorax.

The cover 602 can comprise a material having an elasticity that allows for enough deformation to load the cover 602 into the thoracoscopic delivery device but is rigid enough to withstand the expansion pressure of the diseased tissue. Exemplary materials for the cover 602 include but are not limited to polyurethane, polyester, silicone, and other suitable materials. The cover 602 can have a volume that is configured to accommodate a small amount of expansion of the diseased tissue such that the expansion of the diseased tissue is limited in part by the bounds of the cover 602.

The fastener 600 can be any of the fasteners disclosed herein. In some embodiments the fastener 600 is a band, such as an O-ring. In some embodiments the fastener 600 is relatively stiff such that the opening defined by the fastener 600 has a relatively fixed cross-sectional area. In some embodiments, the opening has a cross-sectional area that allows for some air to pass through the fastener 600 into the diseased tissue when the lung is reinflated. In some embodiments the fastener 600 is adjustable.

Unlike the embodiment shown in FIG. 7A, the therapeutic element 100 in FIG. 7B is not assembled in situ and instead is delivered together with the cover 904 (and any other portion of the therapeutic element 100) to the lung. The fastener 600 can be fixed to the cover 602 such that the fastener 600 does not move relative to the cover 602. In some embodiments, including that shown in FIG. 7B, the fastener 600 may be incorporated along a bottom edge of the cover 602. In other embodiments the fastener 600 may be disposed at other locations along the cover 602. As described above with reference to FIG. 7A, the therapeutic element 100 of FIG. 7B leverages the ability of the diseased tissue to trap air and leverages this defect to secure the therapeutic element 100 to the lung.

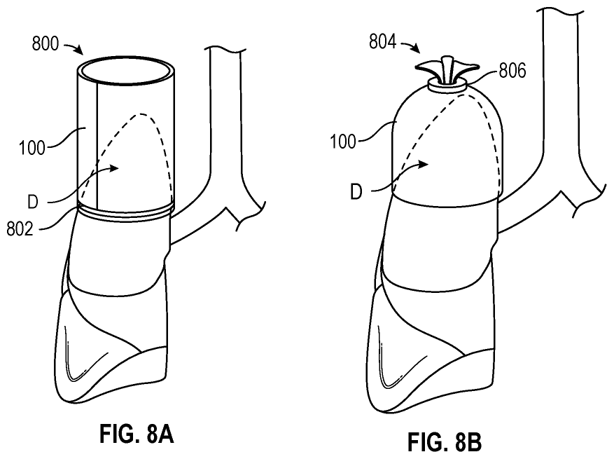

FIGS. 8A and 8B show a therapeutic element 100 in the form of a film (such as film 402) wrapped around diseased tissue D in a collapsed state. In this initial state, the therapeutic element 100 has an end portion 800 that is open and exposing the diseased portion D to the pleural cavity (and thus is not sealed). As shown in FIG. 8B, the system can include a fastener 806 configured to be positioned around the open end portion to close the end portion, thereby limiting or preventing the passage of air between the pleural cavity and the diseased portion and limiting or preventing herniation of the diseased portion. The closed end 804 creates an airtight seal and prevents herniation of the collapsed lung, even after reinflation of the rest of the lung. Additionally or alternatively, the fastener can comprise a thicker portion of the therapeutic element 100 that generates friction with the lung tissue at a magnitude that is greater than a magnitude of friction generated between the rest of the therapeutic element 100 and the lung tissue. For example, with reference to FIG. 8A, the therapeutic element 100 may be secured by using a thicker portion 802 of the therapeutic element 100 to create increased friction during and after the wrapping process. In some embodiments, the therapeutic element 100 can be secured at a level of the lung (such as the lobar fissure) using the thicker portion 802 of the film at its lower edge as it is wrapped.

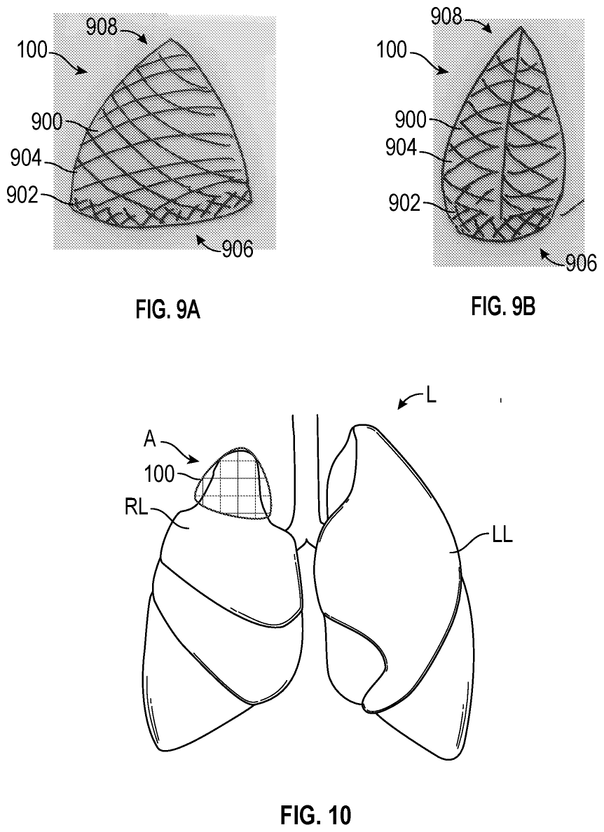

FIGS. 9A and 9B are front and side views of a therapeutic element 100 comprising a mesh envelope having a triangular shape in one dimension to represent the shape of the collapsed apical or upper aspect of the upper lobe of the lung. In the other dimension, the configuration is slightly convex to conform to the collapsed lung. The mesh envelope can be formed of a cut metal stent and/or a braid. In some embodiments, the mesh envelope comprises a superelastic material (e.g., nitinol, a cobalt chromium allow, etc.) and is configured to be thoracoscopically delivered to the chest cavity in a collapsed, low-profile state and expanded once in the cavity to be positioned over the diseased tissue. In some embodiments, the mesh envelope optionally includes a cover 904 disposed across the pores of the mesh (on an inner surface, outer surface, or both). The cover 904 advantageously enables containment of possible air leakage from the collapsed lung, thereby decreasing the chance of a clinically significant pneumothorax.

As shown in FIGS. 9A and 9B, the mesh envelope can comprise a porous sidewall defining a cavity therein and having an open end portion 906 continuous with the cavity and a closed end portion 908. The cavity is configured to receive the diseased portion of the lung therein. The sidewall can comprise a lower portion 902 and an upper portion 900. The lower portion 902 of the mesh envelope can be of greater strength (or of less compliance) to enable tissue compression and to prevent the mesh envelope from being displaced during re-expansion of the lung. For example, in some embodiments the lower portion 902 has a smaller pore size and/or greater strut and/or filament density than that of the upper portion 900 such that the lower portion 902 is more rigid than the upper portion 900. In some embodiments, the upper portion 900 and the lower portion 902 comprise separate meshes that are coupled to one another prior to implantation or coupled to one another in situ. In such embodiments, the lower portion 902 can be less compliant than the upper portion 900.

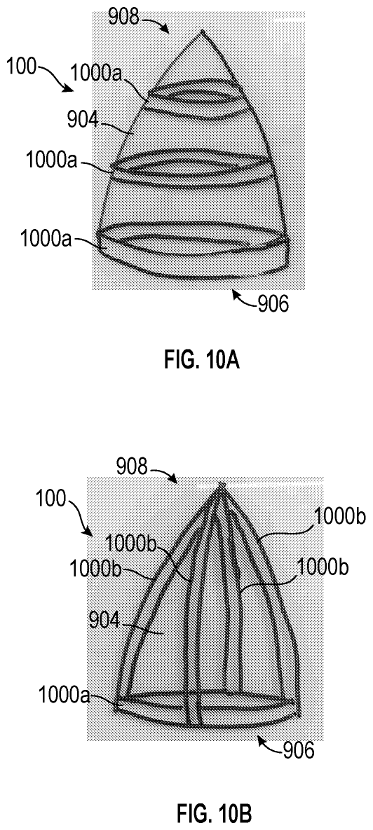

When positioned around and secured to the lung tissue, for example as shown in FIG. 10, the mesh envelope can be biased to assume a flattened configured, which can force the collapsed tissue into a similarly flattened configuration. This flattened shape can prevent or limit twisting of the contained portion of the lung upon reinflation of the remainder of the lung (which can be a challenge for the more cylindrical contained shapes). The flattened shape also advantageously distributes the compressive load across lobe to limit or avoid contact stress and risk of perforation.

FIG. 10A shows a variation of the embodiment shown in FIGS. 9A and 9B. The therapeutic element 100 can have a triangular shape in one dimension to represent the shape of the collapsed apical or upper aspect of the upper lobe of the lung. In the other dimension, the configuration is slightly convex to conform to the collapsed lung. The therapeutic element 100 can comprise a frame and a cover 904. The frame can comprise a plurality of annular struts 1000a that are spaced apart along a height of the therapeutic element 100 and a cover 904 disposed between the struts 1000a (on an inner surface, outer surface, or both). At least one of the struts 1000a is positioned at a lower edge of the therapeutic element 100 (e.g., defining opening 906). The cover 904 advantageously enables containment of possible air leakage from the collapsed lung, thereby decreasing the chance of a clinically significant pneumothorax. In some embodiments of the present technology, the frame comprises a superelastic material (e.g., nitinol, a cobalt chromium allow, etc.) and is configured to be thoracoscopically delivered to the chest cavity in a collapsed, low-profile state and expanded once in the cavity to be positioned over the diseased tissue. Although the therapeutic element 100 of FIG. 10A comprises three annular struts 1000a, in other embodiments the therapeutic element 100 can have more or fewer annular struts 1000a.

FIG. 10B shows another variation of the embodiment shown in FIGS. 9A and 9B. The therapeutic element 100 can have a triangular shape in one dimension to represent the shape of the collapsed apical or upper aspect of the upper lobe of the lung. In the other dimension, the configuration is slightly convex to conform to the collapsed lung. The therapeutic element 100 can comprise a frame and a cover 904. The frame can comprise an annular strut 1000a at the lower edge of the therapeutic element 100 (e.g., defining opening 906), a plurality of longitudinally extending struts 1000b that are laterally spaced apart around the body of the therapeutic element 100, and a cover 904 disposed between the struts 1000a, 1000b (on an inner surface, outer surface, or both). The longitudinal struts 1000b can extend distally away from the annular strut 1000a and come together at a closed end portion 908 of the therapeutic element 100. The cover 904 advantageously enables containment of possible air leakage from the collapsed lung, thereby decreasing the chance of a clinically significant pneumothorax. In some embodiments of the present technology, the frame comprises a superelastic material (e.g., nitinol, a cobalt chromium allow, etc.) and is configured to be thoracoscopically delivered to the chest cavity in a collapsed, low-profile state and expanded once in the cavity to be positioned over the diseased tissue. Although the therapeutic element 100 of FIG. 10B comprises four longitudinal struts 1000b, in other embodiments the therapeutic element 100 can have more or fewer longitudinal struts 1000b. In some embodiments, the therapeutic can have a plurality of annular struts 1000a and a plurality of longitudinal struts 1000b.

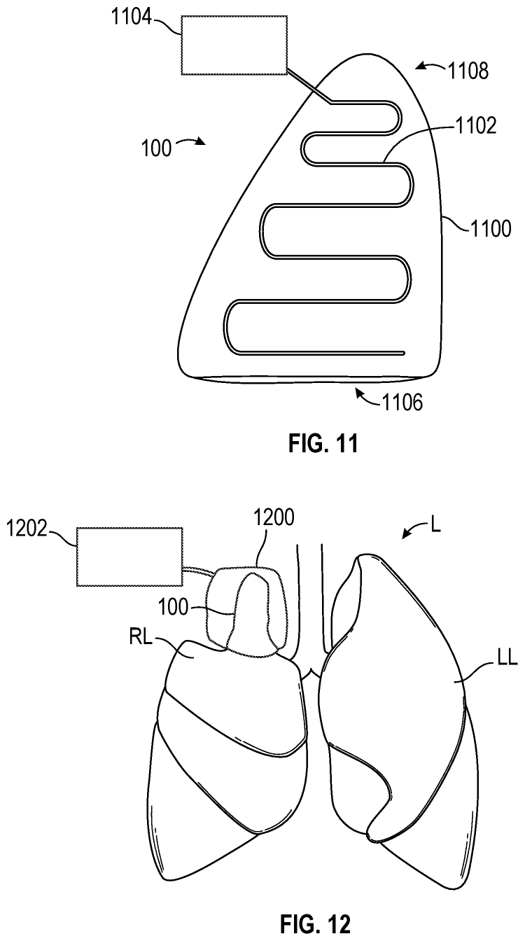

According to some aspects of the present technology, a shape and/or size of the therapeutic element can be adjusted in situ. For example, as shown in FIG. 11, the therapeutic element 100 can comprise a containing structure 1100 configured to be positioned around a portion of the lung and a channel 1102 incorporated with the containing structure 1100. The system can include a fluid source 1104, and the channel 1102 can be configured to be fluidly coupled to the fluid source to receive a fluid from the fluid source 1104, such as a liquid (e.g., water, saline, etc.) or a gas. Controlled delivery of the fluid to and withdrawal of the fluid from the channel 1102 adjusts the size and/or shape of the containing structure 1100, thus affecting the amount of pressure exerted on the lung by the containing structure 1100 as well as the fit of the containing structure 1100 on the lung. According to some methods of use, the therapeutic element 100 is positioned on the collapsed lung and a volume of fluid is delivered to the channel to adjust the shape of the containing structure 1100 to a desired configuration and/or pressure. Fluid delivery may also be used to confirm apposition of the therapeutic element 100 with the lung tissue and/or a contact force between the therapeutic element 100 and the lung tissue. When a desired configuration has been reached, the fluid can be evacuated from the channel 1102 and a volume of a substance equal to the volume of evacuated fluid can be delivered to the channel 1102. The substance can be a flowable liquid or gel that is configured to solidify within the channel so that the containing structure 1100 maintains its desired shape. The therapeutic element 100 thus enables in situ customization that can be beneficial for confirming-prior to deploying the solidifying substance—that the force applied to the lung by the therapeutic element 100 will be sufficient to significantly reduce or prevent airflow into the contained portion of the lung without limiting perfusion to harmful levels.

While the channel 1102 is shown in FIG. 11 only on a single side of the containing structure 1100, in other embodiments the channel 1102 can wrap around all or a portion of the containing structure 1100. Moreover, the channel 1102 can define shapes and/or paths other than those shown in FIG. 11.

The containing structure 1100 can comprise a material having sufficient flexibility and/or elasticity to allow compression and expansion of its shape by the delivery/removal of fluid in the channel. In some embodiments the containing structure 1100 comprises a sidewall defining a cavity therein and having an open end portion 1106 continuous with the cavity and a closed end portion 1108. In a fluid-activated state, the sidewall can be biased to assume a flattened configured or a more three-dimensional configuration. As shown in FIG. 11, in some embodiments the containing structure 1100 is configured to assume a conical and/or pyramidal 3D shape when the fluid is delivered to the channel. This conical and/or pyramidal 3D shape can better approximate the native geometry of the portion of the lung contained by the therapeutic element 100 (and thus provide better tissue contact and sealing) and also prevents or limits twisting of the contained portion of the lung upon reinflation of the remainder of the lung (which can be a challenge for the more cylindrical contained shapes). A flattened configuration can also prevent or limit twisting of the contained portion upon reinflation. The flattened shape also advantageously distributes the compressive load across lobe to limit or avoid contact stress and risk of perforation. The activated shape of the containing structure 1100 can also be configured to limit or prevent constriction of the vasculature that supplies blood to the contained portion of the lung. It will be appreciated that the containing structure 1100 can have other non-triangular, non-conical, and/or non-pyramidal shapes or may not comprise a cupped sidewall. For example, in some embodiments the containing structure 1100 can be a wrap or tubular structure.

In some embodiments, the containing structure 1100 is positioned on an inflated or partially inflated lung and a fluid is delivered to the channel 1102 to compress the containing structure 1100 around the lung.

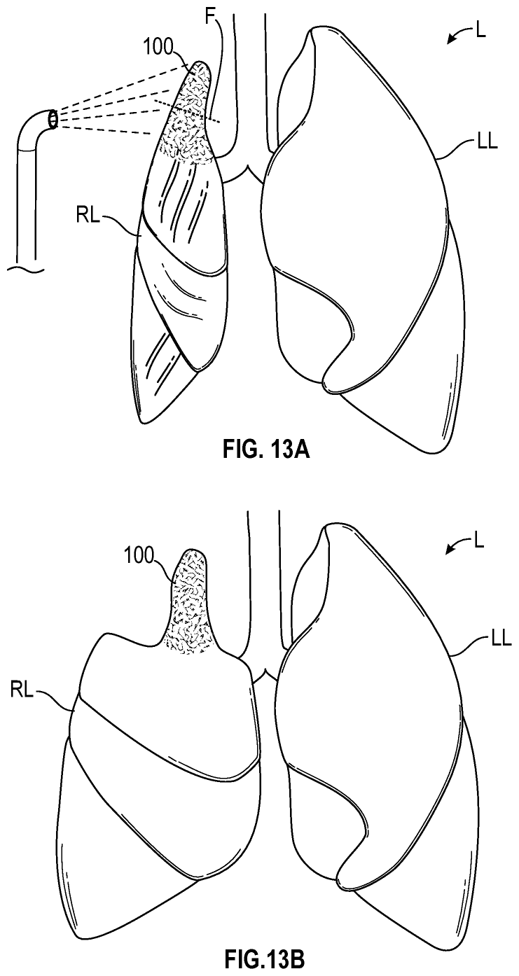

As shown in FIG. 12, in some embodiments the system comprises a therapeutic element 100 and an expandable member 1200 configured to be positioned in the chest cavity with the therapeutic element 100, at least temporarily. The expandable member 1200 can be configured to be fluidly coupled to a fluid source 1202 for controlled expansion and contraction of the expandable member 1200. The expandable member 1200 can fill space in the chest cavity and prevent or limit reinflation of the diseased lobe. In some embodiments, the expandable member 1200 can be inflated until it abuts the chest wall, thereby utilizing the chest wall as an external constraint. According to some methods of use, the expandable member 1200 can be introduced into the chest cavity while the lung is still inflated and may be expanded adjacent the diseased portion of the lung to collapse the diseased portion. The expandable member 1200 can advantageously be expanded gradually (e.g., over a period of hours) to give the targeted portion of the lung time to collapse. Once the lung or targeted portion of the lung is collapsed, the therapeutic element 100 can be applied to hold the collapsed tissue in the collapsed state upon subsequent reinflation of the lung. As detailed herein, in some embodiments the therapeutic element 100 is a solidifying and/or binding substance that can be applied to the collapsed lung tissue and adheres to the surface of the lung and resists expansion of the lung upon reinflation. Regardless of the form of therapeutic element 100 utilized, once it is in place, the expandable member 1200 can be collapsed and removed from the chest cavity to allow the lung to expand into the space previously occupied by the expandable member 1200 while diseased portion is held in a collapsed state by the therapeutic element 100. In some embodiments, the deflation and removal of the expandable member 1200 can be gradual, which may be better tolerated by the tissue.

According to several aspects of the technology, the therapeutic element comprises a flowable solidifying and/or binding substance configured to be administered to the surface of at least the diseased portion of the lung while the lung is in the collapsed state. As depicted in FIGS. 13A and 13B, the flowable solidifying and/or binding substance can be in solution and configured to be administered as a spray and/or coating. In some embodiments, the solidifying and/or binding substance can be a gel. In some embodiments, including that shown in FIGS. 13A and 13B, the flowable solidifying and/or binding substance can be configured to cure, polymerize, and/or solidify in situ to form a substantially rigid film that adheres to the surface of the collapsed, targeted portion of the lung and resists expansion of the lung upon reinflation. Non-mutually exclusive examples of solidifying and/or binding substances include, for example, an adhesive, a polymer, a resin, Progel™, super glue, BioGlue®, and others. In some embodiments, the solidifying and/or binding substance may be UV-curable.

In some embodiments, the solidifying and/or binding substance can be configured to adhere to itself and/or to the surface of the lung. In some cases it may be desirable to apply the solidifying and/or binding substance to the diseased tissue while the diseased tissue is collapsed, thereby forming a first constrained tissue body, then fold the first constrained tissue body over itself (for example, over line F in FIG. 13A) and apply additional solidifying and/or binding substance(s) to secure the first constrained body in a folded over configuration (or “second constrained body”) (not shown). The tissue can be folded and additional solidifying and/or binding substance applied any number of times.

According to some methods of the present technology, a solidifying and/or binding substance can be used in conjunction with a structural member. For example, in some embodiments the structural member is a mesh. The mesh can be disposed on and/or around the diseased portion of the lung, and a solidifying and/or binding substance can be applied to the mesh to secure the mesh to the lung. The mesh can be a fabric, gauze, graft material, braid, stent, stent-graft, or other suitable element. According to some embodiments, a solidifying and/or binding substance in the form of a fluid may be applied to the lung first (for example, by spraying the fluid onto the surface of the lung), and then a structural member can be applied to buttress the solidifying and/or binding substance. In this manner, the presence of the fluid further enhances the fixation of the structural member. The structural member can be, for example, a mesh (as described above), a wrapped film, a cupped sidewall, a tubular sidewall, and/or others. For example, the fluid may be applied to the surface of the lung and the film 402 may be applied and wrapped around the treated portion to buttress the fluid.

According to several aspects of the technology, the system can include one or more sensors, which may be incorporated into or be independent of the therapeutic element. In some embodiments, the one or more sensors include a sensor configured to monitor one or more parameters that characterize the flow of blood to the portion of the lung contained by the therapeutic element. For example, the sensor can be a doppler probe that is configured to detect arterial and venous pulsations consistent with blood flow. It will be appreciated that sensors for monitoring flow other than doppler probes are within the scope of the present technology. Inclusion of a flow sensor can be beneficial for ensuring adequate perfusion to the lung tissue during and/or after installation of the therapeutic element to reduce a risk of ischemia and/or necrosis of the contained portion of the lung. In addition to or instead of a flow sensor, the one or more sensors may include a pressure sensor configured to monitor apposition of the therapeutic element with the targeted portion of the lung and/or a contact force between the therapeutic element and the targeted portion of the lung. The flow sensor, pressure sensor, and any other sensor can be configured to monitor one or more parameters as the therapeutic element is being positioned on the lung and the lung remains collapsed, after the therapeutic element has been installed on the lung and the lung remains collapsed, and/or after the therapeutic element has been installed and the non-contained portions of the lung have been re-inflated.

According to several embodiments, the system can include an imaging device for monitoring the patient following placement of the therapeutic element. The imaging device can be configured for obtaining image data via computed tomography (CT), magnetic resonance imaging (MRI), positron emission tomography (PET), ultrasound, X-ray, or combinations thereof. In some embodiments, the therapeutic element includes an enhancing agent for enhancing visualization of the therapeutic element when imaged by the imaging device. The enhancing agent can be at least one of a sensor, a marker and/or dye.

In any of the embodiments disclosed herein, the therapeutic element can include a therapeutic agent and is configured to deliver the therapeutic agent to the lung tissue while the therapeutic element is secured to the lung tissue. The therapeutic agent can be, for example, at least one of any anti-inflammatory, an antibiotic, and/or an anesthetic. In some embodiments, the therapeutic element comprises a polymeric coating or film that is loaded with the therapeutic agent.

In any of the embodiments disclosed herein, the therapeutic element can comprise a biocompatible material. The biocompatible material can be non-degradable, partially degradable, or fully degradable material. In some embodiments, the biocompatible material comprises a polymeric or biological material. In these and other embodiments, the biocompatible material comprises at least one of silicone, polyurethane, polythioethylene (PTE), polytetrafluoroethylene (PTFE), polyester, nylon, poly(lactic-co-glycolic acid) (PLGA), polylactic acid (PLA), polycaprolactone (PCL), poly(L-lactide-co-F-caprolactone) (PLCL), and all combinations and variations thereof.

In any of the embodiments disclosed herein, the therapeutic element may be of a permanent or bio-absorbable material or a combination thereof.

### CONCLUSION

Although many of the embodiments are described above with respect to systems, devices, and methods for reducing lung volume, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-13B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A system for treating a patient having emphysema via thoracoscopic lung volume reduction, the system comprising:
a therapeutic element having a low-profile configuration for thoracoscopic delivery to the patient's chest cavity and an expanded configuration in which the therapeutic element is configured to be disposed on an outer surface of a diseased portion of a patient's lung; and
an applicator configured to manipulate the therapeutic element within the chest cavity to place the therapeutic element in contact with the diseased portion of the patient's lung when the lung is collapsed,
wherein the therapeutic element is configured to apply a compressive load to and/or restrict expansion of the diseased portion of the patient's lung, and
wherein the therapeutic element is configured to, upon reinflation of the patient's lung, restrict airflow to the diseased portion of the patient's lung, thereby achieving a lung volume reduction, and
wherein interaction between the patient's lung and the therapeutic element produces a contact force and the therapeutic element is configured to distribute the contact force so as to maintain perfusion to the diseased portion of the patient's lung and/or minimize tension in the at least a diseased portion of the patient's lung.

2. The system of claim 1, wherein the therapeutic element comprises a flexible film.

3. The system of claim 1, wherein the applicator comprises a plurality of arms and is configured to transform the therapeutic element from the low-profile configuration to the expanded configuration while the therapeutic element is disposed within the chest cavity.

4. The system of claim 1, wherein the therapeutic element comprises a flexible film, and wherein, when the film is in the low-profile configuration, the film is rolled up.

5. The system of claim 4, wherein the applicator is configured to transform the film from the low-profile configuration to the expanded configuration by unrolling the film.

6. The system of claim 4, wherein the applicator comprises a first arm and a second arm, and wherein the first arm is configured to unroll the film from and the second arm is configured to stabilize the film.

7. The system of claim 1, wherein the therapeutic element comprises a tubular sleeve.

8. The system of claim 1, wherein the therapeutic element is configured to be wrapped around the lung at least one once.

9. The system of claim 1, wherein the therapeutic element is configured to adhere to itself.

10. The system of claim 1, wherein the therapeutic element is configured to be wrapped around the lung at least once and secured via a fastener.

11. The system of claim 10, wherein the therapeutic element is a film and the fastener comprises a thicker portion of the film that generates friction with the lung tissue having a magnitude that is greater than a magnitude of friction generated between the rest of the film and the lung tissue.

12. The system of claim 11, wherein the therapeutic element is configured to be positioned on a lobe of the lung such that the fastener is aligned with a lower edge of the lobe.

13. The system of claim 1, further comprising a fastener, and wherein the fastener is configured to secure the therapeutic element to itself such that the lung tissue is compressively engaged and/or captured within the therapeutic element without the fastener directly engaging the lung tissue, and wherein the fastener comprises at least one of: an adhesive, a mechanical fastener, and/or a weld in situ.

14. The system of claim 1, wherein, when the therapeutic element is positioned over the diseased portion of the lung, the therapeutic element has an end portion that is open and exposing the diseased portion to the pleural cavity, and wherein the system further includes a band configured to be positioned around the open end portion to close the end portion, thereby limiting or preventing the passage of air between the pleural cavity and the diseased portion and limiting or preventing herniation of the diseased portion.

15. The system of claim 1, wherein the therapeutic element is configured to be substantially impermeable to air to seal the lung tissue and prevent leaks.

16. The system of claim 1, wherein the therapeutic element comprises a film and a substance comprising a liquid or gel.

17. The system of claim 16, wherein the substance comprises a biological or polymeric resin and/or glue.

18. The system of claim 16, wherein the substance is configured to be applied to fill interstitial voids between the film and healthy and/or diseased lung tissue to improve attachment and/or sealing between the therapeutic element and the lung tissue.

19. The system of claim 1, wherein the liquid or gel substance is configured to be applied outside the film (i.e., on the other side of the film from the tissue-facing side), and wherein the liquid or gel substance is configured to cure and set the film in a substantially rigid form to restrict expansion of the at least a diseased portion of the patient's lung.

20. The system of claim 1, wherein the therapeutic element is configured to secure the at least a diseased portion of the patient's lung in a substantially flat configuration (i.e., length and/or width are substantially greater than the thickness), and wherein the substantially flat configuration minimizes the contact force and attenuates tension in the lung tissue.

21. The system of claim 1, wherein the therapeutic element comprises a polymer coating configured to adhere to the diseased portion of the patient's lung, and wherein the polymer coating is configured to be sprayed onto the patient's lung when the patient's lung is in a collapsed state.

22. The system of claim 21, wherein the therapeutic element further comprises a biocompatible film, and wherein the film is configured to be applied to and/or wrapped around the polymer coating.

23. The system of claim 22, wherein the film is configured to be a buttress for the polymer coating to enhance fixation and/or sealing of the therapeutic element.

24. The system of claim 1, wherein the therapeutic element includes a therapeutic agent and is configured to deliver the therapeutic agent to the lung tissue while the therapeutic element is secured to the lung tissue.

25. The system of claim 24, wherein the therapeutic agent is at least one of any anti-inflammatory, an antibiotic, and/or an anesthetic.

26. The system of claim 24, wherein the therapeutic element comprises a polymeric coating or film that is loaded with the therapeutic agent.

27. The system of claim 1, wherein the therapeutic element comprises a biocompatible material, wherein the biocompatible material: (a) is either non-degradable, partially-degradable or fully-degradable material, (b) comprises a polymeric or biological material, and/or (c) comprises at least one of silicone, polyurethane, PTE, PTFE, polyester, nylon, PLGA, PLA, PCL (polycaprolactone), PLCL and combinations thereof.

28. The system of claim 1, wherein the therapeutic element is configured for thoracoscopic delivery through at least one port in the wall of the chest of the patent.

29. The system of a claim 1, wherein the system includes a thoracoscope camera.

30. The system of claim 1, wherein the therapeutic element is configured for thoracoscopic delivery via a single port.

31. The system of claim 1, wherein the therapeutic element is configured for thoracoscopic delivery via a multiple ports.

32. The system of claim 1, wherein the therapeutic element comprises a mesh.

33. The system of claim 32, wherein the therapeutic element further comprises a polymer film and/or adhesive configured to secure the mesh to the diseased portion of the lung.

34. The system of claim 1, wherein the therapeutic element comprises a structure configured to be disposed on and around the diseased portion of the patient's lung, wherein the structure includes a channel configured to receive a fluid therethrough.

35. The system of claim 34, wherein a size and/or shape of the structure can be adjusted by controlled delivery of the fluid to and withdrawal of the fluid from the structure, thereby enabling in-situ customization of the therapeutic element.

36. The system of claim 34, wherein upon initial placement of the therapeutic element against the lung tissue, the channel is configured to receive a volume the fluid to confirm apposition of the therapeutic element and the lung tissue and/or a contact force between the therapeutic element and the lung tissue, and, after the fluid is evacuated from the channel, the channel is configured to receive a volume of a substance equal to the volume of fluid, wherein the substance is configured to solidify in-situ and take a permanent shape consistent with the confirmed apposition and/or contact force.

37. The system of claim 34, wherein the structure is configured to assume a triangular two-dimensional (2D) shape and/or a conical and/or pyramidal three-dimensional (3D) shape when the fluid is delivered to the channel to approximate the native geometry of the portion of the lung contained by the therapeutic element, and wherein the 2D and/or 3D shape enables the therapeutic element to prevent or limit twisting of the contained portion of the lung upon re-inflation of the remainder of the lung, and wherein the therapeutic element is configured to limit or prevent constriction of the vasculature perfusing the contained portion of the lung.

38. The system of claim 34, further comprising a substance configured to be delivered into the channel and solidify within the channel to rigidize the structure in a desired shape.

39. The system of claim 1, further comprising a sensor, incorporated into or independent of the therapeutic element, wherein the sensor is configured to monitor perfusion to the portion of the lung contained by the therapeutic element to reduce a risk of ischemia and necrosis of the contained portion of the lung.

40. The system of claim 1, further comprising an imaging device for monitoring the patient following placement of the therapeutic element.

41. The system of claim 40, wherein the imaging device is configured to obtain image data via computed tomography (CT), magnetic resonance imaging (MRI), positron emission tomography (PET), ultrasound, X-ray, or combinations thereof.

42. The system of claim 41, wherein the therapeutic element includes an enhancing agent for enhancing visualization of the therapeutic element when imaged by the imaging device.

43. The system of claim 1, wherein the therapeutic element is configured to have a triangular two-dimensional (2D) shape and/or a conical and/or pyramidal three-dimensional (3D) shape when in an expanded configuration and restricting expansion of the lung.

44. A device for treating a patient having emphysema via thoracoscopic lung volume reduction, the device comprising:
a therapeutic element having a low-profile configuration for thoracoscopic delivery to the patient's chest cavity and an expanded configuration in which the therapeutic element is configured to be disposed on an outer surface of a diseased portion of a patient's lung; and
wherein the therapeutic element is configured to apply a compressive load to and/or restrict expansion of the diseased portion of the patient's lung, and
wherein the therapeutic element is configured to, upon reinflation of the patient's lung, restrict airflow to the diseased portion of the patient's lung, thereby achieving a lung volume reduction, and
wherein interaction between the patient's lung and the therapeutic element produces a contact force and the therapeutic element is configured to distribute the contact force so as to maintain perfusion to the diseased portion of the patient's lung and/or minimize tension in the at least a diseased portion of the patient's lung.

45. The device of claim 44, wherein the therapeutic element comprises a cover.

46. The device of claim 44, wherein the therapeutic element comprises a fastener.
